(19) United States
(12) Patent Application Publication
(10) Pub. No.: US 2005/0064425 A1
(43) Pub. Date: Mar. 24, 2005

(54) Gene function inferring using gene expression data

(76) Inventors: Xinghai Chen, San Diego, CA (US); Guangzhou Zou, San Diego, CA (US); Xun Wang, Chapel Hill, NC (US)

Correspondence Address: JENKINS & WILSON, PA, 3100 TOWER BLVD, SUITE 1400, DURHAM, NC, 27707, US

(21) Appl. No.: 10/500,585

(22) PCT Filed: Feb. 11, 2003
(86) PCT No.: PCT/US03/04324

(30) Foreign Application Priority Data
Feb. 11, 2002 (US) 60355945

(51) Int. Cl. C12Q 1/68; G01V 5/04; G06F 19/00

(52) U.S. Cl. 435/6; 702/10

(57) ABSTRACT

Potential functions of one or more genes are inferred from gene expression measurements. A method involves assembling a distribution of expression level measurements for one or more genes. The method also involves calculating a significance value for each of the expression level measurements. The significance values determine which gene expression samples are classified as relevant and which are classified as irrelevant. The two sample classes are compared to each other to determine the non-overlapping parameters from the gene expression data between them. The non-overlapping parameters are used to indicate gene function of an unknown gene.

### FIELD OF THE INVENTION

The present invention relates to the field of gene function analysis and more particularly, to a method and device for suggesting the function of a gene of unknown function using gene expression data.

### BACKGROUND OF THE INVENTION

The discovery of a gene function can aid in identifying drug targets in the drug development process and further contribute to the basic understanding of biological processes. Conventional methods for determining the function of one or more genes are typically time consuming and costly. An example of such a time consuming and costly method is the creation of “knockout” organisms. For any given gene a “knockout” organism lacking the gene can be created. The resultant “knockout” organism is then observed. Any functional changes observed are then attributed to the knocked out gene.

Information about the possible function of a new gene of unknown function may also be determined by comparing the nucleotide sequence of the unknown gene in one organism to the nucleotide sequences of known genes with known functions in other organisms.

Gene expression levels for a given gene may also offer clues as to the function of the gene. Gene expression levels can be observed by determining the amount of mRNA in for example, a cell, tissue, or organism, under a set of experimental conditions, such as for example, different support medium. Typically, the expression level of an unknown gene is compared to the expression level of genes with known functions. The function of the unknown gene can be determined by selecting the gene or genes of known function with the most similar expression levels. It is assumed that genes with similar expression levels have similar functions. Unfortunately, the conditions under which the gene expression levels are measured are far from uniform. In addition, information regarding the function of known genes is imperfect.

The time and expense to determine the function of even a single gene using existing technology can be substantial. In view of the above, there clearly is a need for an alternative way to determine the function of one or more genes with unknown function.

### SUMMARY OF EMBODIMENTS OF THE INVENTION

A distribution of expression level measurements for one or more genes over a variety of experimental conditions, e.g., parameters, is used in a method and a system to infer the function of a gene. A significance score or value is calculated for each of the expression level measurements. The significance score is calculated using an appropriate statistical method. An example of a significance score includes for example a z-score. A z-score indicates how far and in what direction a data point is from the mean of the distribution as expressed in standard deviation units. A z-score is an example of a simple statistical method that can be used to infer gene function.

The expression level measurements, once assigned a significance value, can be sorted by their significance scores. The step of sorting the expression level measurements is performed according to the significance value calculated for each of the expression level measurements. Measurements within a predefined significance value, for example, having a predefined z-score range, are removed from consideration.

A search is performed on the expression level measurements that are a predefined distance away from the mean of the distribution. For example, expression level measurements that are a predefined number of standard deviation units away from the mean. The search determines which parameters associated with the selected expression level measurements do not overlap with the parameters of the expression level measurements not selected based on their significance value. The search determines which parameters are unique in the selected expression level measurements. The search identifies those significant relevant parameters, which can then help identify functions of the genes. Experimental conditions, as represented by the parameters, are then identified based on significant differences. The identification may be performed in a parameter space of experimental conditions between selected conditions and non-selected conditions. The unique parameters can provide clues to the possible biological functions and utilities of the gene under consideration. The non-overlapped parameters preferably are attributes that are significant or relevant. A function of the one or more genes is suggested based on the non-overlapped parameters.

In one embodiment, the method is implemented as a computer program for determining a function of one or more genes. The program comprises executable instructions that cause a computer to perform the method. The computer program is stored on one of many different types of computer readable media.

### BRIEF DESCRIPTION OF THE DRAWINGS

The invention is pointed out with particularity in the appended claims. The advantages of the invention described above, as well as further advantages of the invention, are better understood by reference to the following detailed description taken in conjunction with the accompanying drawings, in which:

FIG. 1 is a block diagram of an exemplary embodiment of a system that infers gene function according to the present invention;

FIG. 2 is a flowchart illustrating an exemplary embodiment of a process for inferring gene function according to an embodiment of the present invention; and

FIG. 3 is a graph illustrating an exemplary distribution of expression levels according to an embodiment of the present invention.

### DETAILED DESCRIPTION OF THE INVENTION

In the following description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural, logical and electrical changes may be made without departing from the scope of the present invention. The following description is, therefore, not to be taken in a limited sense, and the scope of the present invention is defined by the appended claims.

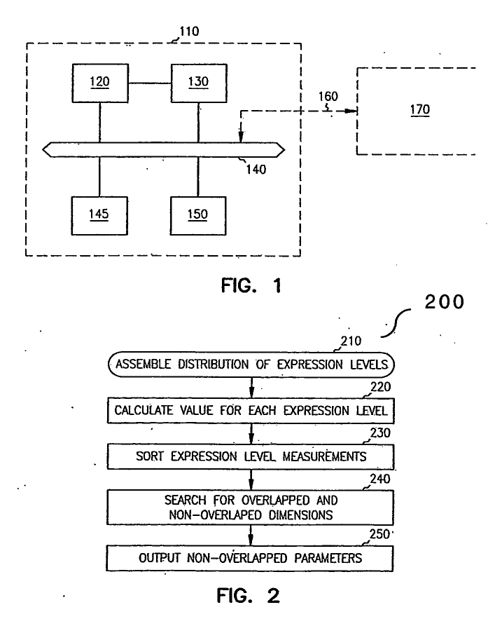

One embodiment for executing a computer program for inferring gene function by searching a gene expression database is illustrated in FIG. 1. A computer system 110 comprises a personal computer or other computer capable of executing computer programs. FIG. 1 is a simplified representation of the computer system 110 comprising a processor 120, a memory 130 and bus 140. The computer system further comprises circuitry and programming for input devices 145 and output devices 150. Input devices 145 can include one or more of disk drives, keyboards, touchpads, and other devices for providing information to the processor 120 and memory 130. Output devices 150 can include one or more of printers, displays, and other output connections.

In one embodiment, computer system 110 has a communications link 160 that is coupled to a network. A data device 170 such as a database server is one device that is also coupled to the network, and hence to the computer system 110 via link 160. The computer system queries the database and receives results from the database. In some embodiments, the communication link is a local or wide area network. In further embodiments, the database server functions are provided by processor 120 utilizing input/output devices 145, 150 such as a disk drive.

Although illustrated as connected via the bus 140, the components of the system 110 may be connected directly to each other in addition to or instead of being connected via the bus 140. Other conventional methods of communicating between components (e.g., conventional wireless communications means) may also be employed. Furthermore, various levels of integration between components may also be contemplated by the present invention. For example, any component may be integrated in part or in whole with any other component or components.

A process, referred to as algorithm 200 and shown in FIG. 2, is used to infer gene function. Software for implementing the process can be stored in computer readable medium such as memory 130 or other input/output devices of the computer system 110. Furthermore, the algorithm 200, in one embodiment, is encompassed in software, hardware (e.g., an application specific integrated circuit (ASIC)) or some combination thereof. The software comprises one or more modules for accomplishing various functions. Each module may be organized as desired to perform single or multiple functions. In further embodiments, selected functions of the algorithm are performed by a human, such as inferring function from non-overlapped parameters identified by the algorithm.

A database stores information or data in an organized fashion, for example, it may store information regarding various gene expression experiments. The database may be stored, at least partly, in the memory 130, the input output devices 145, 150, data device 170, or some combination thereof. Furthermore, the data device 170 may provide additional computing power that can process at least portions of the database stored in the data device 170.

In FIG. 2, a flowchart shows an exemplary embodiment of the algorithm 200 that infers gene function, such as from a vertical search of a database according to an embodiment of the present invention. The algorithm 200 begins at 210, assembling a distribution of expression level measurements for one or more genes. At 220, a significance value is calculated for each of the expression level measurements using an appropriate statistical method. The significance value is representative of the expression level. At 230, the expression level measurements are sorted. At 240, searches are performed for overlapped and non-overlapped dimensions. At 250, the non-overlapped parameters are provided as output and represent potential traits associated with one or more genes.

An example illustrating the operation of the algorithm according to the present invention will be described. The example is to be construed merely as an illustration and is not to be construed as a limitation in any manner.

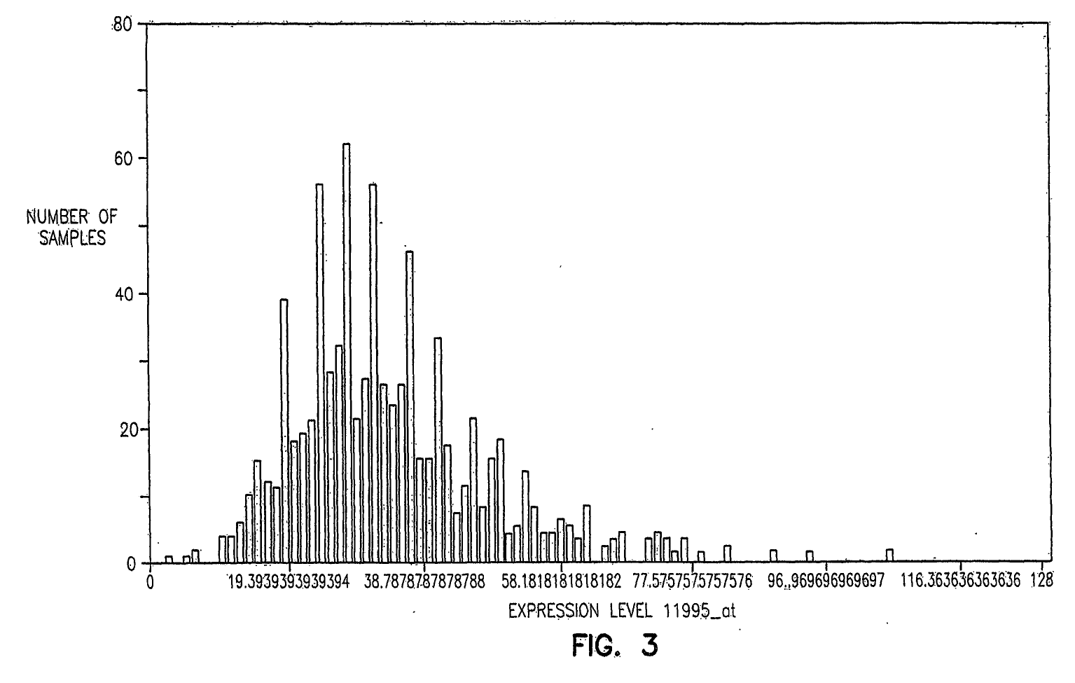

1. The distribution of the gene expression levels is calculated from the database for a given gene or list of genes. For example, an arabidopsis gene expression database consists of more than 1000 measurements for over 8000 genes under diversified conditions defined by over 300 parameters such as, for example, ecotype, tissue, RNA type, harvest conditions, genotype, growth conditions, growth media, treatments, etc. The distribution of the expression levels over the parameter space is obtained for a given gene. For example, the distribution of the expression levels (and therefore the corresponding parameters) for a probset identified as “11995_at” is shown in FIG. 3.

2. A significance score is calculated using an appropriate statistical method, such as, for example, a z-score, for every expression level and therefore for every corresponding sample condition, i.e., experimental condition. The significance score is representative of the distance each expression level is from the mean of the expression levels. Many different statistical methods are available that can provide a significance score.

3. The expression level data and their associated significance score can then be optionally sorted according to significance level, i.e., standard deviation units. For example, when the z-score is the significance score, the expression level data above and below a predefined z-score, for example a z-score greater than 3 and less than −3, are selected. In the probset 11995_at the samples selected from the Arabidopsis gene expression database are:

Sample 00295: (Exp Level 105; ZScore 4.88) Arabidopsis, Columbia, Tissue total, Harvest at 4 pm, 8 hr after treat, seedling, Wild type, Grown in Growth Chamber, Light treated.

Sample 00259: (Exp Level 94; ZScore 4.14) Arabidopsis, Columbia, Tissue leaf, Harvest at 4 pm, 8 hr after treat, seedling, Wild type, Transgenic cDNA B3S sense, Grown in Growth Chamber, Light treated.

Sample 00260: (Exp Level 89; ZScore 3.80) Arabidopsis, Columbia, Tissue total, Harvest at 8 pm, 12 hr after treat, seedling, Wild type, Transgenic cDNA B3S sense, Grown in Growth Chamber, Light treated.

Sample 00263: (Exp Level 78; ZScore 3.07) Arabidopsis, Columbia, Tissue total, Harvest at 8 am, 24 hr after treat, seedling, Wild type, Transgenic cDNA B3S sense, Grown in Growth Chamber, Light treated.

Sample 00266: (Exp Level 82; ZScore 3.34) Arabidopsis, Columbia, Tissue total, Harvest at 8 pm, 36 hr after treat, seedling, Wild type, Transgenic cDNA B3S sense, Grown in Growth Chamber, Light treated.

Sample 00268: (Exp Level 82; ZScore 3.34) Arabidopsis, Columbia, Tissue total, Harvest at 4 am, 44 hr after treat, seedling, Wild type, Transgenic cDNA B3S sense, Grown in Growth Chamber, Light treated.

Sample 00990: (Exp Level 128; ZScore 6.42) Arabidopsis, Ler, Tissue seedling, seedling, Grown in Field.

Each of these samples contains multiple parameters, or experimental conditions under which the sample is processed. For example, the parameters for sample 00990 are—Arabidopsis, Ler, Tissue seedling, seedling, Grown in Field.

4. A search for the non-overlapped/overlapped dimensions in the experimental conditions between the selected significant samples (shown above) and the rest of the samples that are not selected (not shown). In the probset 11995_at the selected sample parameters (shown above) with the sample parameters that were not selected (not shown). The most significant difference (non-overlap) parameter is the light treatment. Many common database algorithms are available to perform such an analysis.

5. The non-overlap parameters are output as the attributes that are significant and the rest of the parameters are irrelevant. In the example of probset 11995_at, the light-treatment is the significant relevant parameter and suggests that the gene has a significant chance to be a clock specific gene. In other words, the gene likely operates in accordance with a circadian cycle, corresponding to a 24 hour cycle of darkness and light.

This process may be repeated for each desired gene in the database.

Although one example has been provided, it is understood that the present invention need not be so limited. For example, although FIG. 2 illustrates a particular order of steps, the present invention may also contemplate other orderings and groupings. In addition, the present invention may include fewer or more steps than illustrated in FIG. 2. For example, sorting of the z-scores is not required, but provides an efficient way of determining the desired z-scores without directly comparing each z-score to the selected thresholds. The present invention contemplates that a process may be formed from a subset of the steps illustrated in FIG. 2. In another example, the present invention may provide additional steps not illustrated in FIG. 2.

Moreover, the significance score is not limited to the z-score and can be selected from any statistical methodology, including for example, any statistical method based on permutations of T-tests, F-tests, nonparametric methods, or combinations thereof. Further, the distribution of the expression data does not have to follow a normal distribution. The statistical methodology chosen should operate to generate a criteria to separate and group at least one member in a data set from the other members in the data set. Furthermore, the method and system of the present is not limited to inferring the function of genes but can also be used to infer the function of other biological molecules such as, for example, proteins, provided there is a large enough amount of data so the statistical methodology can be applied. An example of a sufficient data set size is 1,000 data measurements done under highly diversified conditions and weighted correctly. Preferably, the data points are measured simultaneously. For example, protein chips or DNA microarray chips can be used. Additionally, the method and system of the present invention is not restricted to the use of gene expression data to infer gene function.

One skilled in the art will appreciate that the present invention can be practiced by other than the preferred embodiments which are presented in this description for purposes of illustration and not of limitation, and the present invention is limited only by the claims which follow. It is noted that equivalents for the particular embodiments discussed in this description may practice the invention as well.

In general, it should be emphasized that the various components of embodiments of the present invention can be implemented in hardware, software, or a combination thereof. In such embodiments, the various components and steps would be implemented in hardware and/or software to perform the functions of the present invention. Any presently available or future developed computer software language and/or hardware components can be employed in such embodiments of the present invention. For example, at least some of the functionality mentioned above could be implemented using C or C++ programming languages.

Thus, it is seen that systems and methods for clustering data are provided. One skilled in the art will appreciate that the present invention can be practiced by other than the preferred embodiments which are presented in this description for purposes of illustration and not of limitation and that numerous changes in the details of construction and combination and arrangement of processes and equipment may be made without departing from the spirit and scope of the invention, and the present invention is limited only by the claims that follow. It is noted that equivalents for the particular embodiments discussed in this description may practice the present invention as well.

1. A method of inferring a function of one or more genes, the method comprising:
assembling a distribution of gene expression level measurements, wherein each of said gene expression level measurements comprises at least one parameter;
calculating a significance score for each gene expression level measurement in said distribution;
selecting at least one gene expression level measurement from said distribution based on its significance score;
comparing said at least one parameter of said selected at least one gene expression level measurement to said at least one parameter of non-selected gene expression level measurements; and
determining said at least one parameter from said selected at least one gene expression level measurement that is not present in said non-selected gene expression level measurements.

2. The method of claim 1, wherein said gene expression level measurements are accessed from a database comprising gene expression level measurements for a plurality of genes.

3. The method of claim 1, wherein said significance score indicates a measure of the distance said gene expression level measurements are from a mean value of said gene expression level measurements.

4. The method of claim 3, wherein the significance score is a z-score.

5. The method of claim 4, wherein selected said at least one gene expression level measurement has a z-score greater than 3 and less than −3.

6. The method of claim 3, wherein said at least one gene expression level measurement is selected based on its significance score being a predefined distance from said mean value of said gene expression level measurements.

7. The method of claim 1, wherein said at least one parameter comprises at least one of ecotype, tissue, RNA type, harvest conditions, genotype, growth conditions, growth media, treatments, and combinations therein.

8. The method of claim 1, further comprising sorting the expression level measurements based on said significance scores of said gene expression level measurements.

9. The method according to claim 1, further comprising inferring the function of said one or more genes based on said at least one parameter from said selected at least one gene expression level measurement determined not to be present in said non-selected gene expression level measurements.

10. A computer readable medium having instructions for causing a computer to perform a method of inferring a function of one or more genes, the method comprising:
assembling a distribution of gene expression level measurements, wherein each of said gene expression level measurements comprises at least one parameter;
calculating a significance score for each gene expression level measurement in said distribution;
selecting at least one gene expression level measurement from said distribution based on its significance score;
comparing said at least one parameter of said selected at least one gene expression level measurement to said at least one parameter of non-selected gene expression level measurements; and
determining said at least one parameter from said selected at least one gene expression level measurement that is not present in said non-selected gene expression level measurements.

11. The computer readable medium claim 10, wherein said gene expression level measurements are accessed from a database comprising gene expression level measurements for a plurality of genes.

12. The computer readable medium of claim 10, wherein said significance score indicates a measure of the distance said gene expression level measurements are from a mean value of said gene expression level measurements.

13. The computer readable medium of claim 12, wherein the significance score is a z-score.

14. The computer readable medium of claim 13, wherein selected said at least one gene expression level measurement has a z-score greater than 3 and less than −3.

15. The computer readable medium of claim 12, wherein said at least one gene expression level measurement is selected based on its significance score being a predefined distance from said mean value of said gene expression level measurements.

16. The computer readable medium of claim 10, wherein said at least one parameter comprises at least one of ecotype, tissue, RNA type, harvest conditions, genotype, growth conditions, growth media, treatments, and combinations therein.

17. The computer readable medium of claim 10, further comprising sorting the expression level measurements based on said significance score of said gene expression level measurements.

18. A system for inferring a function of one or more genes, the method comprising:
means for assembling a distribution of gene expression level measurements, wherein each of said gene expression level measurements comprises at least one parameter;
means for calculating a significance score for each gene expression level measurement in said distribution;
means for selecting at least one gene expression level measurement from said distribution based on its significance score;
means for comparing said at least one parameter of said selected at least one gene expression level measurement to said at least one parameter of non-selected gene expression level measurements; and
means for determining said at least one parameter from said selected at least one gene expression level measurement that is not present in said non-selected gene expression level measurements.

19. The system of claim 18, wherein said gene expression level measurements are accessed from a database comprising gene expression level measurements for a plurality of genes.

20. The system of claim 18, wherein said significance score indicates a measure of the distance said gene expression level measurements are from a mean value of said gene expression level measurements.

21. The system of claim 20, wherein said significance score is a z-score.

22. The system of claim 21, wherein said at least one gene expression level measurement selected has a z-score greater than 3 and less than −3.

23. The system of claim 20, wherein selected said at least one gene expression level measurement is based on its significance score being a predefined distance from said mean value of said gene expression level measurements.

24. The system of claim 18, wherein said at least one parameter comprises at least one of ecotype, tissue, RNA type, harvest conditions, genotype, growth conditions, growth media, treatments, and combinations therein.

25. The system of claim 18, further comprising sorting the expression level measurements based on said significance scores of said gene expression level measurements.

26. The system according to claim 18, further comprising inferring the function of said one or more genes based on said at least one parameter from said selected at least one gene expression level measurement determined not to be present in said non-selected gene expression level measurements.

27. A computer system useful for inferring functions of genes, the computer system comprising:
a processor;
a memory coupled to the processor;
a display coupled to the processor;
a computer program that executes on the processor, the program further comprising:
a module assembling a distribution of gene expression level measurements, wherein each of said gene expression level measurements comprises at least one parameter,
a module calculating a significance score for each gene expression level measurement in said distribution,
a module selecting at least one gene expression level measurement from said distribution based on its significance score,
a module comparing said at least one parameter of said selected at least one gene expression level measurement to said at least one parameter of non-selected gene expression level measurements, and
a module determining said at least one parameter from said selected at least one gene expression level measurement that is not present in said non-selected gene expression level measurements.

28. A method of inferring a function of one or more genes, the method comprising:
accessing multiple expression level measurements for multiple samples collected under diversified conditions defined by parameters corresponding to a gene;
calculating a score for the expression level measurements;
selecting samples based on most significant score;
determining non-overlapped parameters between the selected samples and the sample that were not selected; and
inferring gene functions from the non-overlapped parameters.

29. A method for inferring the function of a biological molecule, said method comprising:
assembling a distribution of data points from experimental measurements, wherein each of said data points comprises at least one parameter;
calculating a significance score for each data point in said distribution;
selecting at least one of said date points based on its significance score;
comparing said at least one parameter of said selected at least one data point to said at least one parameter of non-selected data points; and
determining said at least one parameter from said selected at least one data point that is not present in said non-selected data points.

30. The method according to claim 29, further comprising inferring the function of said biological molecule based on said at least one parameter from said selected at least one data point determined not to be present in said non-selected data points.
